SEQUENCE LISTING (5 sequences)

SEQ ID NO: 1 - DNA, 1836 bp, Artificial Sequence
  recombinant DNA
atgggatttgtgttgtttagtcaattgccttcatttcttcttgtgtctactcttttgctttttcttgttattagtcattcatgtagagcacagggtcaaaaggtgtttacaaatacatgggctgtaagaatacctgggggacctgctgttgcaaattctgtggctagaaagcatggttttcttaatttgggtcagatttttggagattattatcatttttggcataggggagtgactaagaggtcactttcaccacataggccaaggcatagtagacttcagagagagcctcaagttcagtggcttgaacaacaagtggctaagagacggactaagagggatgtgtatcaagaaccaacagatcctaagtttccacaacagtggtatctttctggtgttactcagagagaccttaatgttaaggcagcttgggctcagggatatacaggacatggaattgttgtgtctattcttgatgatggaattgaaaagaatcatccagatttggctggaaattatgatccaggtgcttcatttgatgttaatgatcaagatccagatcctcagccaaggtatactcagatgaatgataatagacatggaactaggtgtgcaggtgaggttgctgctgtggctaataacggggtttgtggtgtgggagtggcatataatgctaggattgggggcgttagaatgcttgatggtgaagtgactgatgcagttgaagctaggtctcttggacttaatccaaatcatattcatatctattctgcaagttggggtcctgaagatgatggaaagacagttgatggaccagctaggcttgctgaggaagcattcttcaggggagtgtcacagggtaggggtggattgggttctatttttgtttgggcatctggaaacgggggtagggaacatgatagttgtaattgtgatggatatacaaatagtatatacactctttctattagttcagcaactcaatttggtaatgtgccttggtatagtgaagcatgtagtagtacacttgcaactacatatagttcaggtaatcaaaacgagaagcagattgtgactacggaccttagacaaaagtgtactgagtcacatactggtacttctgcatctgctcctttggcagctggaattattgcacttacacttgaggcaaataagaatcttacatggagggatatgcaacatttggttgttcaaacaagtaagccagcacatcttaatgctaatgattgggcaactaatggtgtgggtagaaaggtgtctcatagttatggatatggtttgcttgatgctggagctatggttgcacttgctcagaattggacaactgtggctcctcagagaaagtgtattattgatattcttactgaacctaaggatattgggaaaagacttgaggttagaaagactgtgacagcatgtttgggtgagccaaatcatattactaggcttgaacacgctcaagctaggttgacacttagttataatagaaggggagaccttgcaattcatcttgtgtctcctatgggaactaggtctactcttttggcagctagaccacatgattatagtgcagatggttttaatgattgggctttcatgactacacattcatgggatgaagatccttctggtgagtgggtgcttgagattgaaaatacatctgaagcaaataactatggaacacttactaagtttacacttgtgctttatggtactgcacctgaaggacttccagtgccacctgagtcttcaggatgtaagacacttacaagtagtcaggcacatcatcatcatcatcataaggatgagctttga

SEQ ID NO: 2 - protein, 611 aa, Artificial Sequence
  recombinant protein
MGFVLFSQLPSFLLVSTLLLFLVISHSCRAQGQKVFTNTWAVRIPGGPAVANSVARKHGFLNLGQIFGDYYHFWHRGVTKRSLSPHRPRHSRLQREPQVQWLEQQVAKRRTKRDVYQEPTDPKFPQQWYLSGVTQRDLNVKAAWAQGYTGHGIVVSILDDGIEKNHPDLAGNYDPGASFDVNDQDPDPQPRYTQMNDNRHGTRCAGEVAAVANNGVCGVGVAYNARIGGVRMLDGEVTDAVEARSLGLNPNHIHIYSASWGPEDDGKTVDGPARLAEEAFFRGVSQGRGGLGSIFVWASGNGGREHDSCNCDGYTNSIYTLSISSATQFGNVPWYSEACSSTLATTYSSGNQNEKQIVTTDLRQKCTESHTGTSASAPLAAGIIALTLEANKNLTWRDMQHLVVQTSKPAHLNANDWATNGVGRKVSHSYGYGLLDAGAMVALAQNWTTVAPQRKCIIDILTEPKDIGKRLEVRKTVTACLGEPNHITRLEHAQARLTLSYNRRGDLAIHLVSPMGTRSTLLAARPHDYSADGFNDWAFMTTHSWDEDPSGEWVLEIENTSEANNYGTLTKFTLVLYGTAPEGLPVPPESSGCKTLTSSQAHHHHHHKDEL

SEQ ID NO: 3 - protein, 30 aa, Artificial Sequence
  Nicotiana benthamiana recombinant protein
MGFVLFSQLPSFLLVSTLLLFLVISHSCRA

SEQ ID NO: 4 - protein, 4 aa, Artificial Sequence
  synthetic sequence
KDEL

SEQ ID NO: 5 - protein, 6 aa, Artificial Sequence
  synthetic sequence
HHHHHH

(19) United States
(12) Patent Application Publication
(10) Pub. No.: US 2021/0292730 A1
(43) Pub. Date: Sep. 23, 2021

(54) IN VIVO POST-TRANSLATIONAL PROCESSING OF TARGET PROTEIN BY FURIN IN PLANTS: ENGINEERING, EXPRESSION AND PRODUCTION OF FUNCTIONAL ACTIVE HUMAN FURIN IN N. BENTHAMIANA PLANTS

(71) Applicant: Akdeniz Universitesi, Antalya (TR)

(72) Inventor: Tarlan MAMMEDOV, Antalya (TR)

(21) Appl. No.: 17/259,616

(22) PCT Filed: Feb. 27, 2019
(86) PCT No.: PCT/TR2019/050123

§ 371 Date: Jan. 12, 2021

(30) Foreign Application Priority Data
Jul. 12, 2018 (TR) 2018/09932

(51) Int. Cl. C12N 9/64; C12N 9/24; C12N 9/80

(52) U.S. Cl. CPC C12N 9/6454; C12Y 304/21075; C12N 9/2402; C12Y 302/01096; C12N 9/80; C12Y 304/21069; C12N 9/6437; C12Y 304/21021; C12Y 304/21022; C12N 9/644; C12N 9/6464; C12Y 305/01052

(57) ABSTRACT

Materials and methods of in vivo possessing of target proteins in plants by co-expressing with proprotein processing enzyme, human Furin, are provided. A method of expressing highly soluble and functional active human Furin in plants also is provided.

### TECHNICAL FIELD THAT THE INVENTION RELATES

This document relates to material and methods of in vivo possessing of target proteins in plants by co-expressing with proprotein processing enzyme, human Furin. A method of expressing highly soluble and functional active human Furin in plants also is provided.

### STATE OF THE ART

Numerous studies in recent years demonstrated plant transient expression systems as the most promising and attractive technology for the production of a variety of recombinant proteins, including vaccine antigens, therapeutic proteins, antibodies, and industrial enzymes. Plant based transient expression system has high expression capacity and provide safe, fast, inexpensive production of valuable recombinant proteins of interest within a short time frame. Furthermore, because plants have eukaryotic post-translational modifications, the technology may be particularly useful for the expression of complex proteins, including mammalian proteins, enzymes, where post translational modifications (PTMs) are critical for the proper folding and functional activity. Although there are significant similarities in mammalian and plant cell PTMs, however, plants lack a number of important mammalian PTMs, including furin (paired basic amino acid cleaving enzyme) cleavage, which does not occur in plants (Wilbers et al., 2016). Furin is a cellular endoprotease, proteolytically activates large numbers of proprotein substrates in secretory pathway compartments by cleaving at clusters of basic residues typically of the form RX(K/R)R. In addition, furin has a vital role in various cellular and bacterial toxin activation (e.g. anthrax and Pseudomonas) and viral propagation (e.g. avian influenza, Ebola fever and human immunodeficiency virus) by activating pathogenic agents and also has a role in cancer metastasis, dementia etc. It should be noted that Furin is one of the modified proteins of Factor IX. Defects in Factor IX (FIX) synthesis result in hemophilia B (Christmas disease), an X-linked disorder. Currently, patients with hemophilia B are mainly treated with Factor IX obtained from concentrates made from human blood and recombinant FIX produced in CHO cell. However, such FIX preparations are extremely expensive and difficult to obtain, especially in developing countries. To date, all attempts at producing recombinant Factor IX using different expression systems have been hampered by limitations in post-translational modification (PTM), safety and high cost. Yet, there remains an urgent need for a safe and affordable therapeutic for hemophilia B. Plant transient expression system could be ideal alternative expression system for the production of safe and affordable recombinant FIX for hemophilia B treatment. As mentioned above, FIX is expressed as a precursor polypeptide that requires posttranslational processing, which lack in plants, to yield a mature protein. Therefore, in order to produce a functional active FIX in plants, FIX must be in vivo processed by furin. In addition, as mentioned above, furin also has a crucial role in so many different cellular events and in diseases. For example, the envelope proteins of viruses of influenza, HIV, dengue fever and several filoviruses including ebola and marburg virus must be cleaved by furin or furin-like proteases to become fully functional. Also, anthrax toxin, pseudomonas exotoxin, and papillomaviruses must be processed by furin during their initial entry into host cells. Bacillus anthracis Protective antigen (PA) has become the main target for anthrax vaccine development. For example, production of heptamerized form of PA63 in plants could be promising alternative to plant produced monomer forms of PA83, which were recently produced and characterized in N. benthamiana plant as a vaccine candidates against anthrax (Mamedov et al. 2016; Mamedov et al., 2017). However, to produce the heptamerized form of PA 63 in plants in vivo, PA83 must be cleaved by functionally active human furin in plants. Thus, production functional active human furin in plant has a potential for many applications. Although plant produced Endo H deglycosylated form of PA83 has increased stability compared to glycosylated or a PNGase deglycosylated PA83 counterparts (Mamedov, Application number: PCT/IB2015/058781; Mamedov et al, 2017), however, still further improvement in the potency and stability of the vaccine candidate is necessary to significantly decrease costs for emergency treatment, especially in case of mass immunization. In this study, the efficient procedure to produce non-N-glycosylated recombinant heptamerized PA63 in N. benthamina plant in vivo by co-expression of target protein, PA83, with modified enzymes, human Furin and deglycosylation enzymes, PNGase F or Endo H in a single cells, was developed. It should be noted that a heptamerized, deglycosylated PA63 can be produced in vitro by the cleavage of recombinant deglycosylated PA83 by commercial available furin. However, given that the commercial recombinant furin used in the cleavage of PA83 is costly, and further purification of cleaved fragment (PA63) is necessary, and therefore, all these steps make the whole process less commercial. At this point, in the current invention, we developed an efficient procedure to produce non-N-glycosylated recombinant heptamerized PA63 in N. benthamina plant in vivo by co-expression of target protein, PA83, with modified enzymes, human Furin and deglycosylation enzymes PNGase F or Endo H in a single cells. Thus, production of functional active human furin in plants could have many potential applications.

### AIM OF THE INVENTION

The aim of the invention is to achieve in vivo processing of target proteins of interest in plants by co-expressing with human Furin.

Another aim of the invention is to engineer human furin for production in N. benthamiana plant and for use in vivo processing of mammalian complex proteins in plant cells; and to confirm that engineered human furin gene produce in vitro or in vivo functional active human furin in N. benthamiana plant.

Another aim of the invention is to develop a strategy for producing the recombinant, deglycosylated, heptamerized form of PA63 in plant cells means by co-expression of PA83 with the processing enzymeurin and deglycosylating enzymes PNGas F or Endo H.

Recombinant factors VII, VIII, IX, and protein C have become important pharmaceuticals in treatment of hemophilia, traumatic bleeding complications and sepsis. In vivo processing of vitamin K-dependent coagulation Factors, such as Factor FIX, Factor VII and protein C in plant cells by furin has not been accomplished. In this study, we engineered human furin gene for expression in plants and demonstrate the expression of highly soluble and functional active truncated form of recombinant human furin in N. benthamiana plant, for the first time.

### DESCRIPTION OF THE FIGURES

FIG. 1 Shows western blot analysis of human furin produced in Nicotiana benthamiana plant.

FIG. 2 Shows a western blot analysis of different dilutions of IMAC purified, plant produced recombinant human Furin.

FIG. 3 SDS-PAGE analysis of recombinant APRIL protein, cleaved by plant produced or commercial Furin.

FIG. 4 Demonstrates in vitro activity assessment of partially purified plant produced Furin by the SDS-PAGE.

FIG. 5 Demonstrates in vitro activity assessment of partially purified plant produced Furin by Western blot analyses.

FIG. 6 Demonstrates in vivo cleavage of FIX by plant produced furin.

FIG. 7 In vivo cleavage of different versions of PA83 by plant produced furin.

FIG. 8 Co-expression of Furin with deglycosylated enzymes Endo H or PNGase F in vivo.

FIG. 9 Effect of deglycosylation of furin on its activity. SDS-PAGE analysis of deglycosylated PA83, treated with deglycosylated plant produced furin in vitro.

### DESCRIPTION OF THE INVENTION

Plant transient expression platform is most promising technology for the production of vaccine antigens, therapeutic proteins, antibodies and industrial enzymes. However, plants do not have some important PTMs, for example. furin processing, which limit this system for the expression of certain mammalian complex proteins. In this study, for the first time, we engineered human furin gene for expression in plants and demonstrate the expression of highly soluble and functional active truncated form of recombinant human furin in N. benthamiana plant. Our results demonstrates that human furin is fully active in vivo and in vitro and successfully cleaved all the tested target proteins, FIX, PA83 and APRIL protein. We demonstrate that both enzymatic deglycosylation and proteolytic processing of proteins can be achieved in vivo by introducing both deglycosylation or furin cleavage enzymes into a eukaryotic system to produce deglycosylated and furin cleaved target proteins. In this study, we also developed a strategy to produce recombinant heptamerazed form, non-glycosylated PA63 in plants by co-expression of PA83 with furin and deglycosylated enzymes, PNGase F and Endo H. A heptamerzed form of PA63 is expected to be more stable and immunogenic than monomeric form of PA83. At this point, this strategy has a potential to develop plant produced non-glycosylated, heptamerazed form of PA63 as an anthrax vaccine candidate. In addition, this strategy is expected to have many potential applications in molecular farming and to be used to produce subunit vaccines, therapeutic proteins, and antibodies in eukaryotic system, for example, can be used for in vivo cleavage of FIX. Several PTMs required FIX for biological activity, including the cleavage of the 18-amino acid propeptide, which have not been previously accomplished in a plant system.

Plant based transient expression system is a promising technology for the production of various recombinant proteins including vaccine antigens, therapeutic proteins, antibodies and industrial enzymes. Although there are significant similarities in mammalian and plant cell PTMs, however, plants lack a number of important mammalian PTMs, including furin cleavage. Furin is one of the modified proteins of vitamin K-dependent coagulation Factors (Factors VII, IX and protein C). Defects in Factor IX (FIX) synthesis result in hemophilia B (Christmas disease), an X-linked disorder. Currently, patients with hemophilia B are mainly treated with Factor IX obtained from concentrates made from human blood and recombinant FIX produced in CHO cell. However, such FIX preparations are extremely expensive and difficult to obtain, especially in developing countries. To date, all attempts at producing recombinant Factor IX using different expression systems have been hampered by limitations in post-translational modification (PTM), safety and high cost. Yet, there remains an urgent need for a safe and affordable therapeutic for hemophilia B. Plant transient expression system could be ideal alternative expression system for the production of safe and affordable recombinant FIX for hemophilia B treatment. As mentioned above, FIX is expressed as a precursor polypeptide that requires posttranslational processing, which lack in plants, to yield a mature protein. Therefore, in order to produce a functional active vitamin K-dependent coagulation Factors (factors VII, IX and protein C) in plants, these coagulation Factors must be cleaved in vivo by PACE/furin processing enzyme. In this study we confirmed that a truncated form of human Furin, produced in N. benthamiana is highly soluble and fully active in vivo and in vitro, and activity comparable with commercial recombinant human furin, and finally successfully specific cleave FIX in vivo. Thus, this strategy has a potential for the production of safe and affordable recombinant FIX in plants for hemophilia B treatment.

Since recombinant human furin was not previously produced in plants, our results support the utility of plants as an expression system for production of active, endotoxin-free recombinant human Furin at reduced costs.

The invention discloses the method and steps to produce functional active human furin in plants and to achieve in vivo processing of target proteins of interest in plants by co-expressing with human Furin as the following:
a first nucleic acid comprising a first nucleotide sequence encoding a human Furin, wherein the first nucleotide sequence is operable linked to a promoter such that when the promoter is activated, the Furin polypeptide is expressed;
second nucleic acid comprising a second nucleotide sequence encoding the polypeptide of interest, wherein the first nucleotide sequence is operable linked to a promoter such that when the promoter is activated, the polypeptide of interest is expressed;
co-expressing the first nucleic acid and the second nucleic acid in the eukaryotic cell, especially; plant cell or yeasts cell to generate the furin prosessed polypeptide,
   wherein the plant cell is a Nicotiana bethamiana cell,

Eukaryotic cell in the method for producing the related furin cleavaged polypeptide and in the method for producing a related N-deglycosylated and furin processed polypeptide is a plant cell.

The first nucleotide sequence in the method for producing the related furin cleavaged polypeptide exhibits at least 90 percent of similarity to the sequence given in SEQ ID NO: 1.

The Furin polypeptide in the method for producing the related furin cleavaged polypeptide exhibits an amino acid sequence with at least 90 percent sequence identity to the sequence set forth in SEQ ID NO:2,

The first, second nucleic acids are introduced into the cell via an Agrobacterium construct and the second nucleotide sequence is vitamin K-dependent coagulation Factors, Factor FIX, Factor VII and protein C.

Method for producing a related N-glycosylated and furin processed polypeptide is yet disclosed by the following process steps:
a first nucleic acid comprising a first nucleotide sequence encoding a human Furin, wherein the first nucleotide sequence is operable linked to a promoter such that when the promoter is activated, the Furin polypeptide is expressed;
a second nucleic acid comprising a second nucleotide sequence encoding a bacterial Endo H (Endo-β-N-acetylglucosaminidase H, Endo H, EC3.2.1.96) or PNGase F glycopeptide N-glycosidase, EC 3.5.1.52), wherein the first nucleotide sequence is operable linked to a promoter such that when the promoter is activated, the Endo H or PNGase F polypeptide is expressed;
a third nucleic acid comprising a nucleotide sequence encoding the polypeptide of interest, wherein the third nucleotide sequence is operable linked to a promoter such that when the promoter is activated, the polypeptide of interest is expressed;
co-expressing the first nucleic acid, the second nucleic acid and the third nucleic acid in the eukaryotic cell, especially; plant cell or yeasts cell to generate the deglycosylated and furin possessed immunoactive polypeptide.

wherein the plant cell is a Nicotiana bethamiana cell,

wherein the eukaryotic cell is a plant cell,

wherein the first nucleotide sequence has at least 90percent sequence identity to the sequence set forth in SEQ ID NO:1.

wherein the Furin polypeptide has an amino acid sequence with at least 90 percent sequence identity to the sequence set forth in SEQ ID NO:2.

wherein the first, second and third nucleic acids are introduced into the cell via an Agrobacterium construct,

wherein the third nucleotide sequence is Bacillus anthracis PA83 protein,

wherein the third nucleic acid sequence encodes a peptide sequence that when produced in its native species is not glycosylated,

wherein the polypeptide is immunoactive in humans,

Specifically cleaved and deglycosylated the polypeptide of interest, Bacillus anthracis PA83 protein is heptamerized in eukaryotic system including plants and,

Specifically cleaved and deglycosylated the polypeptide of interest, Bacillus anthracis PA83 protein is heptamerized in eukaryotic system including plants, thereby increasing its stability and immunogenicity of modified polypeptide is increased.

Studies and results for the method for producing the related furin cleavaged polypeptide and the method for producing a related N-deglycosylated and furin processed polypeptide are disclosed as examples in the following.

### EXAMPLES

### Example 1—Materials and Methods

Cloning, expression and purification of recombinant human Furin produced in N. benthamiana plants, and evaluation of its cleavage activity in vitro and in vivo. The human furin gene was optimized for expression in N. benthamiana plants (for codon optimization, mRNA stability, etc.) and was synthesized at Biomatik Corporation. To transiently express full length furin in N. benthamiana plants, the signal peptide (amino acids 1-25) was removed from the furin sequence (acc.no.NP_002560), and Nicotiana tabacum PR-1a signal peptide (MGFVLFSQLPSFLLVSTLLLFLVISHSCRA) was added to the N-terminus. In addition, the KDEL sequence (the ER retention signal) and the His6 tag (the affinity purification tag) were added to the C-terminus. The resulting sequence was inserted into pEAQ vector (Sainsbury et al., 2009) using AgeI/XhoI sites to obtain pEAQ-Furin-His6-KDEL. To express truncated furin, 25-595 AA was expressed with PR-1a signal peptide at N-terminal and His6-KDEL at C-terminal. The resulting sequence was inserted into the pEAQ vector using AgeI/XhoI sites to obtain pEAQ-Furin (truncated)-His6-KDEL. pEAQ-Furin-His6-KDEL or pEAQ-Furin (truncated)-His6-KDEL was then introduced into the Agrobacterium tumefaciens strain AGL1.

The resulting bacterial strain was grown in BBL medium (10 g/L soy hydrolysate, 5 g/L yeast extract, 5 g/L NaCl, 50 mg/L kanamycin) overnight at 28° C. The bacteria were introduced by manual infiltration into 6-week-old N. benthamiana plants grown in soil. A wild type Nicotiana benthamiana was grown on soil in a greenhouse of Akdeniz University. Four, five, six and seven days after infiltration, leaf tissue was harvested and homogenized using a mortar and pestle. A wild type Nicotiana benthamiana was grown on soil in a greenhouse of Akdeniz University. After homogenization, the extracts were clarified by centrifugation (13 000 g for 30 min) and analyzed using Western blotting.

Cloning and expression of PNGase F, and PA83 in N. benthamiana. The sequences of PNGase F (Mamedov et al., 2012; Mamedov et al., 2017) and B. anthracis PA (amino acids 30-764, GenBank accession number AAA22637, Mamedov et al., 2012) were optimized for expression in N. benthamiana plants and synthesized by IDT (Coralville, Iowa, USA).

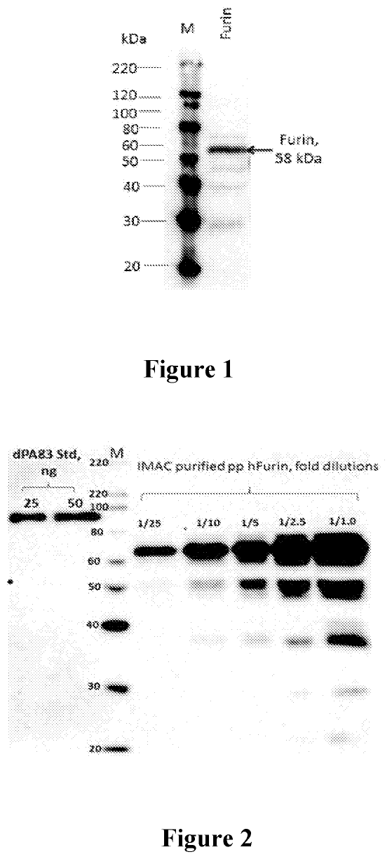

Cloning and expression of Endo in N. benthamiana. The Endo H gene (GenBank accession AAA26738.1) was optimized for expression in N. benthamiana plants and synthesized by GENEART AG (Thermo Fisher Scientific) as described previously (Mamedov et al., 2017). To transiently express Endo H in N. benthamiana plants, the signal peptide (amino acids 1-42) was removed from the Endo H sequence, and Nicotianatabacum PR-1a signal peptide (MGFVLFSQLPSFLLVSTLLLFLVISHSCRA) was added to the N-terminus. In addition, the KDEL sequence (the ER retention signal) and the FLAG epitope (the affinity purification tag) were added to the C-terminus (FIG. 1). The resulting sequence was inserted into the pBI121 expression vectors to obtain pBI-Endo H-FLAG-KDEL.

SDS-PAGE and Western blot analysis. SDS-PAGE analysis of plant produced PA83 samples was performed on 10% acrylamide gels stained with Coomassie (Gel Code Blue, Pierce Rockford, Ill.). Western blot analysis was performed after electrophoresis and transfer of the proteins to Polyvinylidene Fluoride membranes. After transfer, Western blot membranes were blocked with I-Block (Applied Biosystems, Carlsbad, Calif.) and recombinant proteins detected with an anti-4×His (Biolegend) mAb or anti-Bacillus anthracis protective antigen antibody [BAP0101] (ab1988). The membranes were then washed with 1×PBS containing 0.1% Tween 20 (PBS-T) to remove an excess primary antibody and labeled with an anti-mouse horseradish peroxidase (HRP)-conjugated secondary antibody (ab98790) or anti-rabbit horseradish peroxidase (HRP)-conjugated secondary antibody (ab97051). Signal generation was achieved with a chemiluminescent substrate (SuperSignal West Pico, Thermo Fisher Scientific, Grand Island, N.Y.); the membrane was incubated with 5 ml of SuperSignal West Pico Chemiluminescent Substrate for 5 minutes wrapped in plastic then the image was taken using high sensitive GeneGnome XRQ Chemiluminescence imaging system (Syngene, A Division of Synoptics Ltd).

### Example 2—Production of Recombinant Human Furn in N. benthamiana, Purification Using IMAC Column

Furin is enriched in the Golgi apparatus, where it functions to cleave other proteins into their mature/active forms. Furin cleaves proteins just downstream of a basic amino acid target sequence (canonically, Arg-X-(Arg/Lys)-Arg). In addition to processing cellular precursor proteins, furin is also utilized by a number of pathogens. It should be noted that although expression of human furin in N. benthamiana plant was recently reported, nevertheless, there was no report to confirm the expression of human furin in N. benthamiana by Western blot analysis (Wilbers et al., 2016). It has been reported that co-expression of the protease furin in Nicotiana benthamiana leads to efficient processing of latent transforming growth factor-b1 into a biologically active protein (Wilbers et al., 2016), however, there was no reports on its expression and direct production confirmation in plants, for example, by western blot using specific antibody. First, we tried to express the entire length of human furin in N. benthamiana. However, when full length furin was expressed with PA83 protein in N. benthamiana there was little or no cleavage of PA83 protein (data not shown). In addition, western blot analysis showed that when full length furin was expressed in N. benthamiana plant there was little or no specific protein band was detected. Therefore, a truncated form of human Furin was engineered to produce functional active and highly soluble recombinant furin in N. benthamiana plant, using plant transient expression system. Thus, in order to produce a human furin in N. bentamiana plant, the furin gene was optimized using N. bentamiana codons. We demonstrated the expression of human furin in the N. benthamiana plant and confirmed the expression by Western blot analysis (FIG. 1). In order to confirm the functional cleavage activity of human furin in vitro, plant produced human Furin was purified using IMAC column. Western blot analysis of different dilutions of IMAC purified, plant produced recombinant human Furin was demonstrated in FIG. 2. For the assessment of in vitro cleavage activity of plant produced Furin, concentration of furin in IMAC purified samples was quantified using the gene tools software, Syngene Bioimaging. Recombinant APRIL, PA83 and FIX proteins were used to evaluate the in vitro cleavage activity.

### Example 3. Assessment of In Vitro Cleavage Activity of Plant Produced Furin Using APRIL Protein

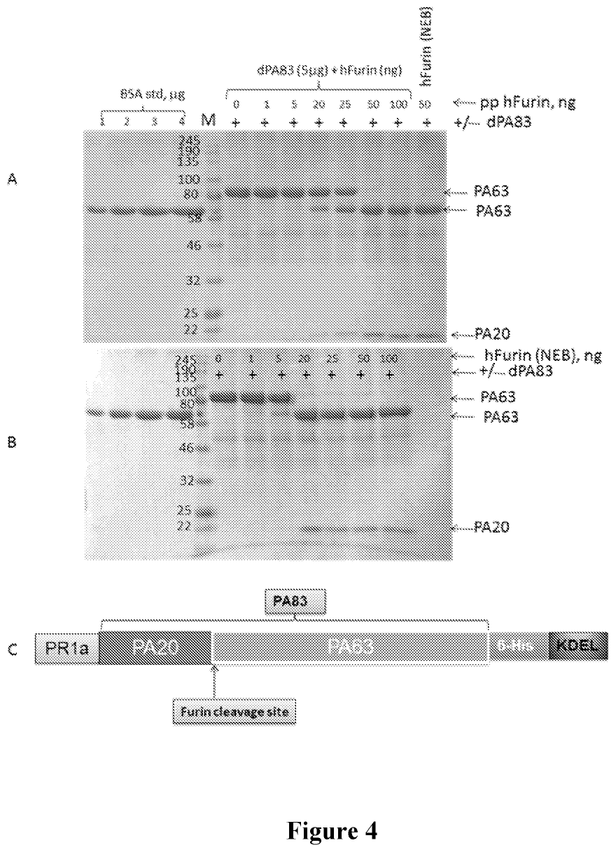

Recombinant murine APRIL is a soluble 21.9 kDa protein and can be cleaved by the protease, furin, to release a soluble C-terminal fragment, which comprises the TNF like receptor binding of the APRIL precursor. Recombinant APRIL from mouse was purchased from Sigma (SRP3189-20UG). In order to test the cleavage activity of plant produced human Furin, 5 μg APRIL recombinant protein were incubated with 25 ng plant produced human Furin or 25 ng commercial human Furin at 25° C., for 1 h. As can be seen from FIG. 4 recombinant APRIL protein is cleaved by both plant produced human Furin (25ng) or commercial Furin (25 ng) and 16.8 kDa fragment was generated. These results confirm that plant produced human furin is enzymatically active in vitro as a commercial (New England Biolabs) human Furin.

### Example 4. Assessment of In Vitro Cleavage Activity of Plant Produced Furin Using PA83 Protein

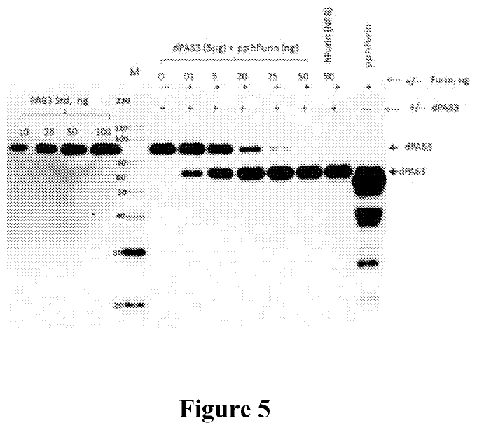

B. anthracis PA is an 83-kDa (PA83) protein that is cleaved to the 63-kDa protein (PA63) by furin at the furin cleavage sequence (Moayeri et al., 2007), IMAC purified PA83 (5 μg) protein was incubated with different concentrations (1, 5, 20, 25, 50 and 100 ng) of IMAC purified furin at 25° C., for 1 h. Protein samples were analyzed by SDS-PAGE and western blot analysis. As shown in FIGS. 4 and 5, by increasing plant produced Furin concentration, the generation of PA63 and PA20 fragments increases and 50 ng the plant produced Furin completely cleaved. 5 μg plant produced PA83 protein. These results confirm that plant produced Furin is fully active in vitro and successfully cleaved PA83 protein, resulting in the generation of PA63 and PA20 fragments.

### Example 5. Assessment of In Vivo Cleavage Activity by Co-Expression with FIX Protein

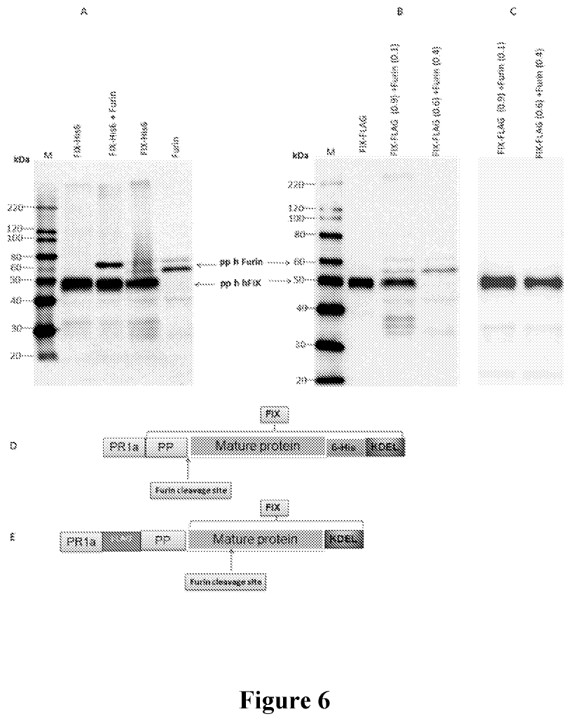

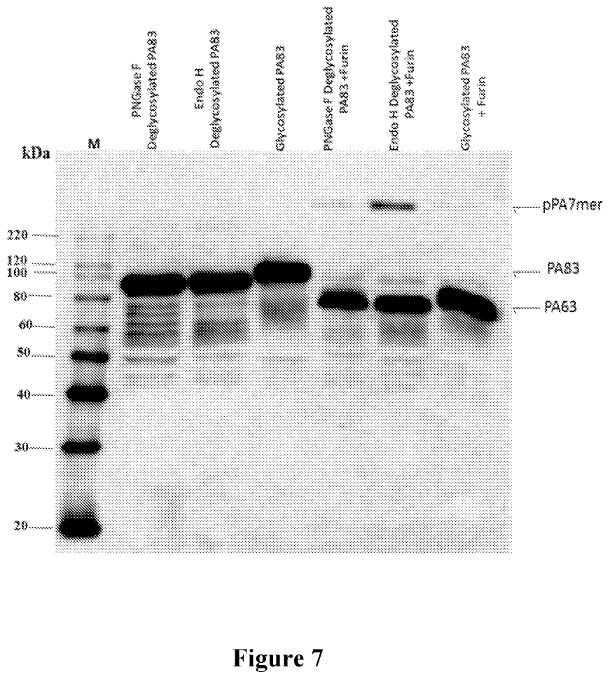

Factor IX is expressed as a precursor polypeptide that requires posttranslational processing to yield a mature protein. In particular, the precursor polypeptide of Factor IX requires Vitamin K-dependent-□□□carboxylation of 12 glutamic acids from the N-terminus and cleavage of propeptide, the 18-amino acid residue sequence N-terminal to the Gla domain. In vivo, GGCX binds to the propeptide which is then cleaved. Propeptide cleavage is required for optimal binding of the Gal domain to Ca⁺⁺ and phospholipids. When overexpressed in CHO cells, furin facilitates propeptide cleavage of FIX even when the recombinant protein is expressed at very high levels (Wasley et al., 1993; Rehemtulla et al., 1992, 1993). Therefore, expression of an active propeptide processing enzyme furin in plants is critical for the production of functionally active FIX in plants. At this point, we performed in vivo co-expression of Factor IX with human furin in N. benthamiana plant for possible cleavage of FIX by plant produced Furin. The results were demonstrated in FIG. 7. As can be seen from FIG. 6 A, since Furin cleavage fragment is a 18-amino acid residue sequence it is very hard to detect the shift of Factor IX, co-expressed with furin. Therefore, we constructed a new FIX sequence, with FLAG epitop at the N-terminal. As can be seen from FIG. 7, when PA83 was co expressed with furin in the OD ration of 0:9 and 0.1, respectively, the FIX protein band is observed. However, in the OD ration of PA83 and Furin is 0:9 and 0.1, respectively, no FIX band was observed, suggesting the lost of FLAG epitop by FIX cleavage. These results demonstrate that plant produced human Furin is functional active in vivo and FIX is correctly cleaved by furin.

### Example 6. Assessment of In Vivo Cleavage Activity by Co-Expression with PA83 Protein

Production of non-glycosylated heptamerized form of PA from Bacillus anthracis in Nicotiana benthamiana by co-expression with human furin and bacterial Endo H or PNGase F.

As mentioned, B. anthracis PA is an 83-kDa (PA83) protein and following binding to receptors, PA83 is rapidly cleaved by cell surface furin to release a 63 kDa (PA63) and 20 kDa polypeptide (PA20) (Gordon and Leppla, 1994; Gordon et al., 1995). Cell-bound PA63 rapidly heptamerize to form a pre-pore (Molloy et al., 1992). Heptamerized PA binds LF or EF and facilitates the exotoxin entry into the cytoplasm that leading to cell death. I hypothesized that by co-expression of PA83 with the deglycosylated enzyme and furin, heptamerized form of PA63 may be generated in vivo. When PA83 co-expressed with human furin and bacterial Endo H or PNGase F, the protein mobility shift on SDS-PAGE was observed with the PA83 protein due to the cleavage by Furin and deglycosylation by Endo H or F PNGase F (FIG. 7). By comparison molecular mass of PA83, PA63 and deglycosylated PA83, it has been determined that PA83 protein undergoes two modifications in vivo, i) is successfully cleaved by plant produced furin and ii) is deglycosylated by plant produced bacterial Endo H or F PNGase F. As shown in FIG. 7, high molecular mass protein band was only observed in plant-produced glycosylated PA83s, indicating heptamerization of furin cleaved PA63 fragment in vivo. It should be noted that high molecular band was barely observed with PNGase F deglycosylated, but not observed with glycosylated PA83, suggesting that heptamer formation of deglycosylated PA83 is probably blocked by plant N-glycans. This is explain why plant produced glycosylated PA83 has no biological activity and could not form LeTx in vitro (Chichester et al., 2013).

### Example 7. Assessment of In Vivo Cleavage Activity by Co-Expression with FIX Protein

Thus, our study demonstrates for the first time that the Bacillus anthracis PA83 is efficiently deglycosylated and specifically cleaved by the plant produced human plant produced Endo H/PNGase F or furin, respectively, in an over-expression system and subsequently, cleaved PA63 is heptamerized. This is a new strategy for production the heptamerized form of deglycosylated PA63 in vivo in a protein expression system, including in plants.

Western blot analysis of human furin produced in N. benthamiana plants is shown FIG. 1. According to FIG. 1, leaf samples were taken at 6 dpi and homogenized in three volumes of extraction buffer. After centrifugation at 13 000 g for 20 min, samples were run on SDS-PAGE prior to Western blotting. M: MagicMark XP Western Protein Standard (ThermoFisher Scientific).

Western blot analysis of different dilutions of IMAC purified, plant produced recombinant human Furin is shown FIG. 2. According to FIG. 2, dPA83 Std-purified plant produced Endo H deglycosylated, purified PA83 that used as a standard protein. Concentration of furin in IMAC purified samples was quantified using the gene tools software, Syngene Bioimaging. M: MagicMark XP Western Protein Standard (ThermoFisher Scientific).

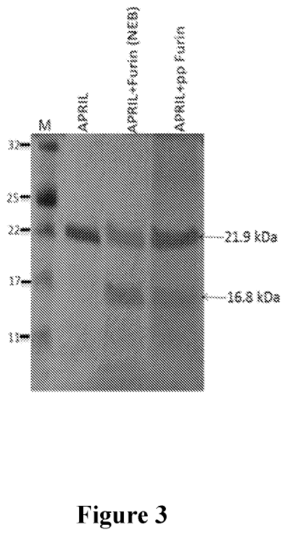

SDS-PAGE analysis of recombinant APRIL protein, cleaved by plant produced or commercial Furin, as indicated is shown FIG. 3. According to FIG. 3, recombinant murine APRIL is a soluble 21.9 kDa protein and can be cleaved by the protease, furin, to release a soluble C-terminal fragment, which comprises the TNF like receptor binding of the APRIL precursor. Recombinant APRIL from mouse was purchased from Sigma (SRP3189-20UG). As can be seen from Fig. recombinant APRIL protein is cleaved by both plant produced human Furin (25 ng) or commercial Furin (25 ng) and 16.8 kDa fragment was generated. These results confirm that plant produced human furin is active in vitro as a commercial (New England Biolabs) human Furin. M: dark color prestained protein standard (New England Biolabs).

SDS-PAGE analysis of dPA83 cleaved with plant produced or commercial human Furin (New England Biolab) is shown FIG. 4. According to FIG. 4, A: 5 μg dPA83 (deglycosylated PA83) samples were treated with different concentrations (0, 1, 5, 20, 25, 50 and 100 ng) of plant produced human Furin or 50 ng commercial (New England Biolabs) human Furin as indicated, and then 4.5 μg samples were run on SDS-PAGE. B: 5 μg dPA83 (deglycosylated PA83) samples were treated with different concentrations (0, 1, 5, 20, 25, 50 and 100 ng) of commercial human Furin (New England Biolabs) as indicated, and then 4.5 μg samples were run on SDS-PAGE.C: Schematic representation of PA83 protein structure. PA63 and PA20 (a 20-kDa amino-terminal fragment) are cleavage products of PA83 by human Furin. M: dark color prestained protein standard (New England Biolabs).

Western blot analysis of dPA83 cleaved with plant produced human Furin is shown FIG. 5. According to FIG. 5, A: 5 μg dPA83 (deglycosylated PA83) samples were treated with different concentrations of plant produced human Furin or 50 ng commercial (New England Biolabs) human Furin as indicated, and then 100 ng samples run loaded into gel. pphuman Furin: plant produced, IMAC purified furin. M: MagicMark XP Western Protein Standard (ThermoFisher Scientific).

Western blot analysis of FIX, in vivo co-expressed with plant produced human Furin is shown FIG. 6. According to FIG. 6, A: samples were loaded as indicated. Proteins on the blot were probed with anti-His antibody. B: samples were loaded as indicated. Proteins on the blot were probed with anti-FLAG antibody. C: Proteins on the blot were probed with anti-FIX antibody. D: schematic representation of FIX-His-KDEL construct structure. E: schematic representation of FLAG-FIX-KDEL construct structure. M: MagicMark XP Western Protein Standard (ThermoFisher Scientific).

PA83 protein was in vivo co-expressed with or without deglycosylation enzymes Endo H or PNGase F or with or without furin is shown FIG. 7. Proteins were probed with anti-His tag antibody and the image was taken using high sensitive GeneGnome XRQ Chemiluminescence imaging system. pPA7 mer, heptameric PA63. M: MagicMark XP Western Protein Standard (ThermoFisher Scientific).

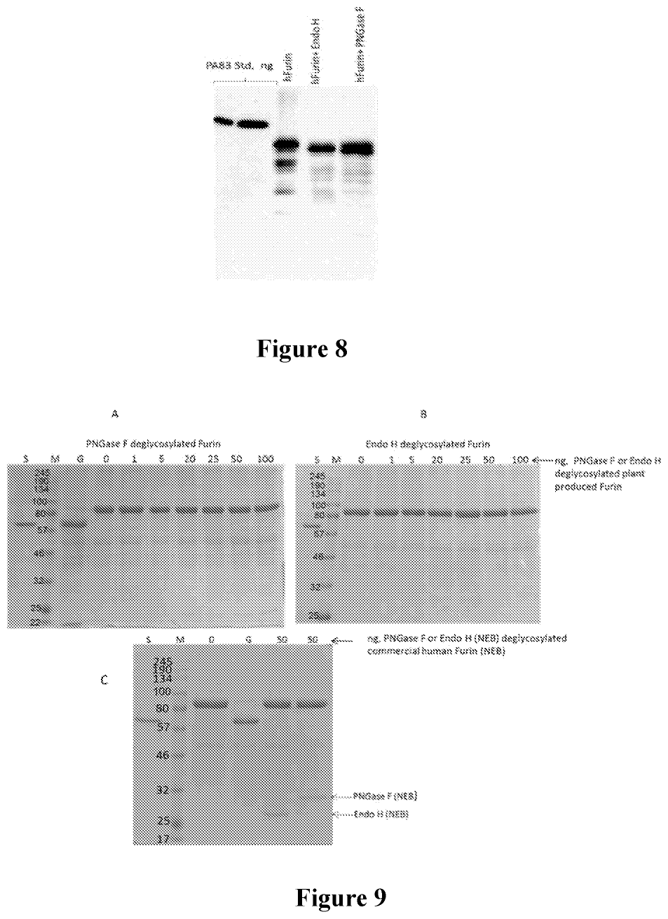

Co-expression of Furin with deglycosylating enzymes Endo H and PNGase F in vivo is shown in FIG. 8. Furin was expressed alone using pEAQ-Furin constructs or with pBI-Endo H or pBI-PNGase F constructs to produce deglycosylated variants of recombinant plant produced Furin. All furin variants, produced in N. benthamiana plant were purified using IMAC column. IMAC column purified proteins were analyzed in Western blotting.

Effect of deglycosylation of furin on its activity. SDS-PAGE analysis of deglycosylated PA83, treated with deglycosylated plant produced furin in vitro is shown in FIG. 9. A: PNGase F deglycosylated furin at different concentrations (0, 1, 5, 25, 50, 100 ng) incubated with 5 □g plant produced deglycosylated PA83 in vitro. B: Endo H deglycosylated furin at different concentrations (0, 1, 5, 25, 50, 100 ng) incubated with 5 □g plant produced deglycosylated PA83 in vitro. C: Commercial Endo H or PNGase F deglycosylated 50 ng commercial human furin incubated with plant produced deglycosylated PA83 in vitro. G: plant produced deglycosylated PA83 was incubated with plant produced non-deglycosylated Furin; S-BSA standard; M: M: color prestained protein standard (New England Biolabs).

In summary, we demonstrate that plant produced furin is highly active, in vitro and in vivo and successfully cleaved of tested proteins at its cleavage site. Recombinant factors VII, VIII, IX, and protein C have become important pharmaceuticals in treatment of hemophilia, traumatic bleeding complications and sepsis. Furin is one of the modified proteins of vitamin K-dependent coagulation factors. Furin also has a vital role in so many different cellular and bacterial toxin activation (e.g. anthrax and Pseudomonas) and viral propagation (e.g. avianinfluenza, Ebola fever and human immunodeficiency virus) by activating pathogenic agents. Plant expression system with eukaryotic post-translational modification machinery, is a promising technology for the production of various recombinant proteins including vaccine antigens, therapeutic proteins, antibodies, native additives and industrial enzymes, and offers superior efficiency, scalability, safety, and lower cost over other expression systems. However, due to lack some postranslational modifications (PTMs) in plants, this expression system may not be a suitable expression platform for some proteins, especially complex human proteins, for example FIX, which require several PTMs for functional activity including Furin processing. In this study, we engineered human furin for expression in plants and demonstrated the expression and production of a truncated form of recombinant human furin in Nicotiana benthamiana plant, for the first time. Our results show that plant produced human furin is highly soluble and fully active in vivo and in vitro and successfully cleaved the tested target proteins, such as FIX, PA83 and APRIL. We also demonstrate that both enzymatic deglycosylation and proteolytic processing of proteins can be achieved in vivo by introducing both deglycosylation or furin cleavage enzymes into a eukaryotic system to produce deglycosylated and furin cleaved target proteins. This technology is expected to have many potential applications in molecular farming and to be used to produce therapeutic proteins and subunit vaccines in eukaryotic system at reduced costs. In addition, since recombinant furin has not been previously expressed in plants, our results support the utility of plants as an expression system for production of an active, endotoxin-free furin at reduced costs.

### INDUSTRIAL APPLICATION OF THE INVENTION

Plant transient expression platform is most promising technology for the production of vaccine antigens, therapeutic proteins, antibodies and industrial enzymes. However, plants do not have some important PTMs, for example, furin processing, which limit this system for the expression of certain mammalian complex proteins.

In this study, we engineered human furin gene for expression in plants and demonstrated the expression of highly soluble, functional active recombinant human furin in N. benthamiana plant, for the first time. Our results demonstrated that human furin is fully active in vivo and in vitro and successfully specifically cleaved the tested target proteins, such as FIX, PA83 and APRIL protein. Furin is one of the modified proteins of vitamin K-dependent coagulation Factors (Factors VII, IX and protein C). Defects in Factor IX (FIX) synthesis result in hemophilia B (Christmas disease), an X-linked disorder. Currently, patients with hemophilia B are mainly treated with Factor IX, obtained from concentrates made from human blood and recombinant FIX produced in CHO cell. However, such FIX preparations are extremely expensive and difficult to obtain, especially in developing countries. To date, all attempts at producing recombinant Factor IX using different expression systems have been hampered by limitations in post-translational modification (PTM), safety and high cost. Yet, there remains an urgent need for a safe and affordable therapeutic for hemophilia B. Plant transient expression system could be ideal alternative expression system for the production of safe and affordable recombinant FIX for hemophilia B treatment. As mentioned above, FIX is expressed as a precursor polypeptide that requires posttranslational processing, which lack in plants, to yield a mature protein. Therefore, in order to produce a functional active vitamin K-dependent coagulation Factors (factors VII, IX and protein C) in plants, these coagulation Factors must be cleaved in vivo by PACE/furin processing enzyme. In this study, we confirmed that a truncated form of human Furin, produced in N. benthamiana plant is highly soluble and fully active in vivo and in vitro, and activity of plant produced furin comparable with commercial recombinant human furin. Thus, this strategy has a potential for the production of safe and affordable recombinant FIX for hemophilia B treatment. Taking together, this strategy could be usefully for expression of functional active vitamin K-dependent coagulation Factors, such as Factor FIX, Factor VII and protein C in plant system. In this study, we also demonstrate that both enzymatic deglycosylation and proteolytic processing of proteins can be achieved in vivo by introducing both deglycosylation and furin processing enzymes into a eukaryotic system to produce deglycosylated and furin processed target proteins. Although PNGase F (Mamedov et al., 2016) or Endo H deglycosylated forms (WIPO Application Number: PCT/IB2015/058781; Mamedov et al., 2016; Mamedov et al., 2017) of PA83 are more stable than the glycosylated counterpart and have a superior immunogenicity as compared to a glycosylated forms, however, still further improvement in the potency and stability of the vaccine candidate is necessary to significantly decrease costs. In this study, we developed a strategy to produce recombinant deglycosylated, heptamerazed form of PA63 in plant cells by co-expression of PA83 with furin and deglycosylated enzymes such as PNGase F and Endo H. It is possible that a heptamerzed form of PA63 could be stable than monomeric form. At this point, this strategy has a potential to develop a non-glycosylated, heptamerazed form of PA63 as a new generation vaccine candidate against anthrax. In addition, this strategy is expected to have many potential applications in molecular farming and to be used to produce subunit vaccines, therapeutic proteins, and antibodies in eukaryotic system. Since recombinant human furin was not previously produced in plants, our results support the utility of plants as an expression system for production of active, endotoxin-free recombinant human Furin at reduced costs.

### REFERENCES

Gordon V M, Leppla S H. Proteolytic activation of bacterial toxins: role of bacterial and host cell proteases. Infect Immun. 1994; 62:333-40.

Chichester J A, Manceva S D, Rhee A, Coffin M V, Musiychuk K, Mett V, et al. (2013) A plant-produced protective antigen vaccine confers protection in rabbits against a lethal aerosolized challenge with Bacillus anthracis Ames spores. Hum Vaccin Immunother 9: 544-552.

Chichester J A, Musiychuk K, de la Rosa P, Horsey A, Stevenson N, Ugulava N, et al. (2007) Immunogenicity of a subunit vaccine against Bacillus anthracis. Vaccine 25: 3111-3114.

Mamedov T, Ghosh A, Jones R M, Mett V, Farrance C E, Musiychuk K, et al. (2012) Production of nonglycosylated recombinant proteins in Nicotiana benthamiana plants by co-expressing bacterial PNGase F. Plant Biotechnol J 10:773-782.

Mamedov T, Chichester J A, Jones R M, Ghosh A, Coffin M V, Herschbach K, Prokhnevsky A I, Streatfield S J, Yusibov V. Production of Functionally Active and Immunogenic Non-Glycosylated Protective Antigen from Bacillus anthracis in Nicotiana benthamiana by Co-Expression with Peptide-N-Glycosidase F (PNGase F) of Flavobacterium meningosepticum. PLoS One. 2016 Apr. 21; 11(4):e0153956

Mamedov T, Cicek K, Gulec B, Ungor R, Hasanova G. In vivo production of non-glycosylated recombinant proteins in Nicotiana benthamiana plants by co-expression with Endo-β-N-acetylglucosaminidase H (Endo H) of Streptomyces plicatus. PLoS One. 2017 Aug. 21; 12(8):e0183589. doi: 10.1371/journal. pone.0183589. eCollection 2017.

Tarlan MAMMEDOV. Production of in vivo N-deglycosylated recombinant proteins by co-expression with Endo H WO 2017081520 A1, PCT/IB2015/058781, 17 May 2017.

Tarlan MAMMEDOV. In vivo de-glycosylation of recombinant proteins by co-expression with PNGase F WO 2012170678 A1, 13 Dec. 2012.

Molloy S S, Bresnahan P A, Leppla S H, Klimpel K R, Thomas G (1992) Human furin is a calcium-dependent serine endoprotease that recognizes the sequence Arg-X-X-Arg and efficiently cleaves anthraxtoxin protective antigen. J Biol Chem 267: 16396-16402.

Wilbers R H, Westerhof L B, van Raaij D R, van Adrichem M, Prakasa A D, Lozano-Torres J L, Bakker J, Smant G, Schots A. Co-expression of the protease furin in Nicotiana benthamiana leads to efficient processing of latent transforming growth factor-β1 into a biologically active protein. Plant Biotechnol J. 2016 August; 14(8):1695-704.

Thomas G. Furin at the cutting edge: from protein traffic to embryogenesis and disease. Nat Rev Mol Cell Biol. 2002, 3:753-66. (2002).

1. A method for producing a Furin to carry out in vivo process of the target proteins in plants by means of co-expressing human furin that is a proprotein processing enzyme, wherein said method comprises:
a first nucleic acid comprising a first nucleotide sequence encoding a highly soluble and functional active truncated form of recombinant human Furin, wherein the first nucleotide sequence is operable linked to a promoter such that when the promoter is activated, the Furin polypeptide is expressed and wherein the first nucleotide sequence has the sequence set forth in SEQ ID NO: 1 or SEQ ID NO: 2,
second nucleic acid comprising a second nucleotide sequence encoding the polypeptide of interest, wherein the first nucleotide sequence is operable linked to a promoter such that when the promoter is activated, the polypeptide of interest is expressed and chosen from any of vitamin K-dependent coagulation Factors, Factor FIX, Factor VII and protein C,
co-expressing the first nucleic acid and the second nucleic acid in the eukaryotic cell, especially; plant cell or yeasts cell to generate the furin processed polypeptide,
wherein the plant cell is a Nicotiana bethamiana cell.

2. A method of producing a N-deglycosylated and Furin processed polypeptide, wherein the method comprises:
a first nucleic acid comprising a first nucleotide sequence encoding a highly soluble and functional active truncated form of recombinant human Furin, wherein the first nucleotide sequence is operable linked to a promoter such that when the promoter is activated, the Furin polypeptide is expressed and wherein the first nucleotide sequence has the sequence set forth in SEQ ID NO: 1 or SEQ ID NO: 2,
a second nucleic acid comprising a second nucleotide sequence encoding a bacterial Endo H (Endo-β-N-acetylglucosaminidase H, Endo H, EC3.2.1.96) or PNGase F glycopeptide N-glycosidase, EC 3.5.1.52), wherein the first nucleotide sequence is operable linked to a promoter such that when the promoter is activated, the Endo H or PNGase F polypeptide is expressed,
a third nucleic acid comprising a nucleotide sequence encoding the polypeptide of interest, wherein the third nucleotide sequence is operable linked to a promoter such that when the promoter is activated, the polypeptide of interest is expressed,
co-expressing the first nucleic acid, the second nucleic acid and the third nucleic acid in the eukaryotic cell, especially; plant cell or yeasts cell to generate the deglycosylated and furin possessed immunoactive polypeptide,
wherein the plant cell is a Nicotiana bethamiana cell.

3. The method according to claim 1, wherein the first, second nucleic acids are introduced into the cell via an Agrobacterium construct.

4. The method according to claim 2, wherein the first, second and third nucleic acids are introduced into the cell via an Agrobacterium construct.

5. The method according to claim 2, where in the third nucleotide sequence is Bacillus anthracis PA83 protein.

6. The method according to claim 2, wherein the third nucleic acid sequence encodes a peptide sequence that when produced in its native species is not glycosylated.

7. The method according to claim 2, wherein the polypeptide is immunoactive in humans.

8. The method according to claim 5, wherein Bacillus anthracis PA83 protein is in heptamerized form.
